(19) United States
(12) Patent Application Publication
(10) Pub. No.: US 2023/0043872 A1
(43) Pub. Date: Feb. 9, 2023

(54) COMPOSITION FOR LIP PAIN RELIEF AND METHOD OF MAKING

(71) Applicant: Meghan Wyscaver, Dexter, MI (US)

(72) Inventor: Meghan Wyscaver, Dexter, MI (US)

(21) Appl. No.: 17/864,695

(22) Filed: Jul. 14, 2022

Related U.S. Application Data
(60) Provisional application No. 63/222,078, filed on Jul. 15, 2021.

(51) Int. Cl. A61K 9/06; A61K 47/44; C12N 9/50; A61K 47/22; A61K 47/14

(52) U.S. Cl. CPC A61K 9/06; A61K 47/44; C12Y 304/22004; C12N 9/63; A61K 47/22; A61K 47/14

(57) ABSTRACT

A soothing lip balm composition includes a wax binder component, which includes at least two compounds selected from a C₈-C₃₀ fatty acid, esterified C₈-C₃₀ fatty acid, and C₈-C₃₀ fatty alcohols; a complex fat component, which includes at least three different compounds selected from C₈-C₃₀ fatty acids; an organic compound component, which includes at least three compounds selected from 2,5-dimethoxy-p-cymene, cumene, thymol methyl ether, 2,6-diisopropylanisole, decanal, and 1,2,2,3-tetramethylcyclopent-3-enol; and an enzymatic component, which includes cysteine endopeptidase.

### CROSS-REFERENCE TO RELATED APPLICATIONS

This application claims the benefit under 35 U.S.C. 119(e) of U.S. Provisional Patent Application No. 63/222,078 filed Jul. 15, 2021, the entire disclosure of which is hereby incorporated herein by reference for any purpose whatsoever.

### BACKGROUND

Lip augmentation or filling is a cosmetic treatment that serves to improve the balance and appearance of the fullness of a person's lips. During a procedure, lidocaine, or another topical anesthetic, is applied to the patient's lip area, and then filler material is injected into the patient's lips and surrounding tissue. Hyaluronic acid gel may be used as the filler material, though fats or collagen are also sometimes used. Results last for several months, but repeat procedures are necessary to maintain the desired appearance.

Pain, swelling, and bruising in the lips and surrounding tissues are common conditions after the procedure. Bacterial infections are also possible. These side effects can be painful and are cited by some patients as their reason for not repeating the procedure. Thus, there is a need for a product that can provide pain relief and reduce the swelling, bruising, and discomfort resulting from cosmetic lip augmentations.

### SUMMARY

The present application is directed to a composition for pain relief, decreased bruising, and decreased swelling due to cosmetic surgery and other types of procedures; and to a method of making the same, and to a method of treating discomfort, pain, swelling, or bruising using the composition. According to some embodiments, the composition includes a wax binder component, a complex fat component, an organic compound component, and an enzymatic component.

In some embodiments, the method includes the steps of a) heating a blend of a wax binder component and a complex fat component to about 175° F. to create a mixture and keeping the mixture at that temperature for at least about 20 minutes; b) maintaining the temperature of the mixture at about 175° F. for at least about 20 minutes, c) reducing the temperature of the mixture to about 150° F.; d) adding an organic compound component and an enzymatic component; e) rapidly cooling the mixture to about 50° F.; wherein the wax binder comprises at least two compounds selected from a C₈-C₃₀ fatty acid, esterified C₈-C₃₀ fatty acid, and C₈-C₃₀ fatty alcohols; the complex fat component comprises at least three different compounds selected from C₈-C₃₀ fatty acids; the organic compound component comprises at least three compounds selected from 2,5-dimethoxy-p-cymene, cumene, thymol methyl ether, 2,6-diisopropylanisole, decanal, 1,2,2,3-tetramethylcyclopent-3-enol, and cannabidiol; and the enzymatic component comprises cysteine endopeptidase.

In another embodiment, the method includes a) heating a mixture of beeswax, shea butter, mango butter, sunflower seed oil, liquid coconut oil, and jojoba heated to about 175° F.; b) maintaining the temperature of the mixture at 175° F. for at least about 20 minutes or until the mixture is homogenized, c) reducing the temperature of the mixture to about 150° F.; d) adding arnica oil and bromelain extract to the mixture; and e) setting the mixture in a freezing environment to rapidly cool to the mixture to about 50° F.

In another aspect, the method includes treating pain including administering to a person in need of treatment a therapeutically effective amount of a composition, wherein the composition includes: a wax binder component comprising at least two compounds selected from a C8-C30 fatty acid, an esterified C8-C30 fatty acid, and a C8-C30 fatty alcohol; a complex fat component comprising at least three different compounds selected from C8-C30 fatty acids; an organic compound component comprising at least three compounds selected from 2,5-dimethoxy-p-cymene, cumene, thymol methyl ether, 2,6-diisopropylanisole, decanal, α-isocomene, and 1,2,2,3-tetramethylcyclopent-3-enol;andan enzymatic component comprising cysteine endopeptidase.

Further embodiments are described in more detail below.

### DETAILED DESCRIPTION

The composition herein disclosed is, according to some embodiments, useful for treating damage to lips and other tissue caused by cosmetic surgery and other types of procedures. It may also have other applications for treating skin and tissue irritated or damaged by other processes or events, such as, for example, other types of surgery or injuries.

One aspect of the present disclosure is a lip salve composition that includes a wax binder component, a complex fat component, an organic compound component, and an enzymatic component. In some embodiments, the lip salve composition includes other optional components to increase the beneficial effects of the composition.

According to some embodiments, the wax binder component comprises at least one compound selected from long chain fatty acids (C₈-C₃₀), esterified long chain fatty acids (C₈-C₃₀), and fatty alcohols (C₈-C₃₀). The wax binder component provides body and texture to the composition, allowing the product to be applied to specific areas of the lips or skin to maximize the beneficial effects. In some embodiments, the wax binder is derived from a natural source, such as nuts, seeds, flowers, or other plant matter. The amount of wax binder may be increased or decreased to achieve the desired product body and texture. In some embodiments, the composition includes about 2 to 40 wt. % of the wax binder component. In other embodiments, the composition includes about 3 to 30 wt. %, about 4 to 20 wt. % and, in some instances, about 5 to 15 wt. % of the wax binder component. In some embodiments, the composition includes about 10 wt. % of the wax binder component. In other embodiments, the composition includes about 6 wt % of the wax binder component.

The complex fat component comprises at least three different compounds selected from C₈-C₃₀ fatty acids. For example, this component may include saturated fatty acids such as caprylic, pelargonic, capric, undecylic, lauric, tridecylic, myristic, pentadecylic, palmitic, margaric, stearic, nonadecylic, arachidic, heneicosylic, behenic, tricosylic, lignoceric acids, pentacosylic, cerotic, carboceric, montanic, nonacosylic, and melissic acids. The complex fat component may also include monounsaturated fatty acids such as caproleic, lauroleic, myristoleic, palmitoleic, oleic, elaidic, vaccenic, gadoleic, erucic, brassidic, or nervonic acid. The complex fat component may also include polyunsaturated fats such as linoleic, columbinic, searidonic, mead arachidonic, eicosapentaenoic, docosapentaenoic, or docosahexaenoic acid. In some embodiments, the complex fat is derived from a natural source, such as nuts, seeds, fruits, leaves, flowers, or other plant matter. The composition may include about 20 to 80 wt. % of the complex fat component. Typically, the composition commonly includes about 30 to 65 wt. %, about 40 to 60 wt. % and, in some instances, or about 45 to 55 wt. % of the complex fat component. In some embodiments, the composition includes about 55 wt. % of the complex fat component. In other embodiments, the composition includes about 80 wt. % of the complex fat component.

The organic compound component includes at least three compounds selected from 2,5-dimethoxy-p-cymene, cumene, thymol methyl ether, 2,6-diisopropylanisole, decanal, α-isocomene, and 1,2,2,3-tetramethylcyclopent-3-enol. These chemical compounds may be derived from a natural source, such as nuts, seeds, fruits, leaves, flowers, or other plant matter. These compounds have shown anti-cancer, anti-inflammatory, anti-oxidative, antibacterial, and/or anti-parasitic benefits. Typically, the composition includes about 0.1 to 20 wt. %, about 0.5 to 18 wt. % and, in some instances, about 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, or 15 wt. % of the organic compound component. In some embodiments, the composition includes about 15 wt. % organic compound component. In another embodiment, the composition includes about 1 wt. % organic compound component. In other embodiments, the composition includes about 5 wt. % organic compound component.

The enzymatic component includes at least cysteine endopeptidase. This compound may be derived from a natural source, such as nuts, seeds, fruits, leaves, flowers, or other plant matter. In some instances, the composition includes about 0.01 to 2 wt. %, about 0.05 to 0.5 wt. % and, in other instances, about 0.1 to 0.25 wt. % of the enzymatic component. In some embodiments, the composition includes about 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, or 2.0 wt. % of the enzymatic component. In some embodiments, the composition includes about 0.1 wt. % cysteine endopeptidase.

In any of the above embodiments, the composition may include optional components to condition or soften the skin, reduce inflammation, provide pain relief, reduce swelling, and to promote the beneficial effects of the composition. These optional components may be derived from a natural source, such as nuts, seeds, fruits, leaves, flowers, or other plant matter. For example, aloe-emodin, aloetic-acid, anthranol, cannabidiol oil (CBD oil), and/or barbaloin may be used. In some embodiments, the composition may include vitamin E oil. In some embodiments, the composition may include vitamin K oil. In some embodiments, the composition may include cannabidiol oil. In some embodiments, the composition may include sambucus nigra fruit extract. In some embodiments, the composition may include elderberry fruit extract. In some embodiments, the composition may include olive oil. In some embodiments, the composition may include menthone, menthofuran, neoisomenthol, menthol, or 1,8-cineole. In some embodiments, menthone, menthofuran, neoisomenthol, menthol, or 1,8-cineole is derived from mint oil. In some of these embodiments, at least a portion of the mint oil is peppermint oil. In some of these embodiments, at least a portion of the peppermint oil is from the mentha x piperita breed of peppermint.

In some embodiments, the wax binder can be provided from a natural source. In some embodiments, the wax binder is beeswax. In other embodiments, the wax binder is soy wax. In other embodiments, the wax binder is candelilla wax. In other embodiments, the wax binder is paraffin wax. In other embodiments, the wax binder is rice bran wax. In other embodiments, the wax binder is sunflower wax. In other embodiments, the wax binder is olive wax. In other embodiments, the wax binder is carnauba wax. In other embodiments, the wax binder is a combination of any two or more of the foregoing waxes.

In some embodiments, the complex fat component is a naturally derived complex fat. In some embodiments, the complex fat component is shea butter. In other embodiments, the complex fat component is mango butter. In other embodiments, the complex fat component is sunflower seed oil. In other embodiments, the complex fat component is liquid coconut oil. In other embodiments, the complex fat component is solid coconut oil. In other embodiments, the complex fat component is jojoba oil. In some embodiments, the complex fat component is a mixture of at least three of the foregoing naturally-derived complex fats. In some embodiments, the complex fat component is a mixture of shea butter, mango butter, sunflower seed oil, liquid coconut oil, and jojoba oil.

In some embodiments, the organic compound component is derived from a natural source. In some embodiments, the organic compound component is arnica oil. In some embodiments, the organic compound is a derivative of the mountain arnica or Arnica montana plant. In other embodiments, the organic compound is a derivative of Arnica chamissonis.

In some embodiments, the enzymatic compound is also derived from natural sources. In some embodiments, the enzymatic compound is bromelain, bromelain extract, pineapple extract, pineapple stem extract, or a combination thereof.

Another aspect of the present disclosure is a method of making a composition for application to a patient's lips after lip filler or other cosmetic surgery. The composition is preferably in the form of a salve, but other forms such as balms, ointments, tinctures, and lotions are also contemplated. At a basic level, the method comprises heating the wax binder and the complex fat components to a first temperature and maintaining the mixture at this elevated temperature for at least about 20 minutes so that the wax binder and complex fat components homogenize. The mixture is then cooled to a second temperature at which the organic compound and enzymatic components are added to the mixture. The lower temperature is used to reduce the damage to the organic compound and enzymatic components. Optional components may also be added at this temperature. Once the organic compound and enzymatic components have been integrated into the mixture, the mixture is rapidly cooled to a third temperature. If desired, the mixture may be poured into other vessels, such as packaging for individual sales, before the rapid cooling step. Alternatively, the cooled mixture may be divided into vessels once the third temperature has been achieved.

The first temperature must be sufficient to melt the wax binder and complex fats and facilitate complete mixing, while being below such a temperature that damage to the mixture, such as protein or fat denaturing or oxidation, begins to occur. In some embodiments, the first temperature is about 150-200° F. In some embodiments, the first temperature is about 175° F. It is important to keep the wax binder and complex fat components at the first temperature to allow the components to mix well, which can help prevent the formation of crystals in the composition when the composition is cooled. The second temperature likewise must be low enough to not denature the proteins added by the organic compound and the enzymatic compound and any optional components. In some embodiments, the second temperature is about 100-175° F. In some embodiments, the second temperature is about 100-150° F. While not wishing to be bound by any particular theory, it is believed that cooling the composition to the third temperature rapidly may avoid the formation of crystals in the composition. Rapid cooling may also prevent separation of the components or cracking in the composition as it cools. Rapid cooling of the composition may be done by exposing the composition to temperatures near or below 32° F. or lower until the mixture reaches 50° F. or lower. In some embodiments, this is done by placing the mixture in a freezer or blast chiller until the composition reaches the third temperature.

To create a composition with optional components, such as elderberry extract, vitamin E, vitamin K, or cannobidiol oil, the optional components may be added to the mixture at the first or second temperature, depending on the nature of the optional components with consideration given to whether the optional components are fat or protein based. Protein based components containing beneficial enzymes or other proteins should be added at temperature low enough as to not harm the protein structure.

Another aspect of the present disclosure is a method of treating pain, swelling, or discomfort associated with cosmetic treatments/surgery. This method may also be employed to address pain, swelling, or discomfort due to other causes as well, such as other surgeries or injuries to the skin or tissue of the body.

In one embodiment, the method includes administering to a person in need of treatment a therapeutically effective amount of any of the compositions heretofore disclosed, including those in which the composition includes: a wax binder component comprising at least two compounds selected from a C₈-C₃₀ fatty acid, an esterified C₈-C₃₀ fatty acid, and a C₈-C₃₀ fatty alcohol; a complex fat component comprising at least three different compounds selected from C₈-C₃₀ fatty acids; an organic compound component comprising at least three compounds selected from 2,5-dimethoxy-p-cymene, cumene, thymol methyl ether, 2,6-diisopropylanisole, decanal, α-isocomene, and 1,2,2,3-tetramethylcyclopent-3-enol; and an enzymatic component comprising cysteine endopeptidase.

Tables A-E, below, provide illustrations of exemplary formulations described herein.

TABLE A
Illustrative Composit ion Formulation

| | Base Formula |
|---|---|
| Ingredient (wt. %) | (wt. %) |
| Beeswax | [symbol] |
| Shea Butter | [symbol] |
| Mango Butter | [symbol] |
| Sunflower Seed Oil | [symbol] |
| Liquid Coconut Oil | [symbol] |
| Jojoba Oil | [symbol] |
| Arnica Oil | [symbol] |
| Bromelain Extract | [symbol] |

TABLE B
Illustrative Composition Formulation

| | Base Formula |
|---|---|
| Ingredient (wt. %) | (wt. %) |
| Beeswax | [symbol] |
| Shea Butter | [symbol] |
| Mango Butter | [symbol] |
| Sunflower Seed Oil | [symbol] |
| Liquid Coconut Oil | [symbol] |
| Jojoba Oil | [symbol] |
| Olive Oil | [symbol] |
| Arnica Oil | [symbol] |
| Bromelain Extract | [symbol] |

TABLE C
Illustrative Composition Formulation

| | Base Formula |
|---|---|
| Ingredient | (wt. %) |
| Beeswax | [symbol] |
| Shea Butter | [symbol] |
| Mango Butter | [symbol] |
| Sunflower Seed Oil | [symbol] |
| Liquid Coconut Oil | [symbol] |
| Jojoba Oil | [symbol] |
| Cannabidiol Oil | [symbol] |
| Arnica Oil | [symbol] |
| Bromelain Extract | [symbol] |

TABLE D
Illustrative Composition Formulation

| | Base Formula |
|---|---|
| Ingredient | (wt. %) |
| Beeswax | [symbol] |
| Shea Butter | [symbol] |
| Mango Butter | [symbol] |
| Sunflower Seed Oil | [symbol] |
| Liquid Coconut Oil | [symbol] |
| Jojoba Oil | [symbol] |
| Elderberry Extract | [symbol] |
| Arnica Oil | [symbol] |
| Bromelain Extract | [symbol] |

TABLE E
Illustrative Composition Formulation

| | Base Formula |
|---|---|
| Ingredient | (wt. %) |
| Beeswax | [symbol] |
| Shea Butter | [symbol] |
| Mango Butter | [symbol] |
| Sunflower Seed Oil | [symbol] |
| Liquid Coconut Oil | [symbol] |
| Jojoba Oil | [symbol] |
| Arnica Oil | [symbol] |
| Bromelain Extract | [symbol] |
| Aloe Vera Gel | [symbol] |
| Vitamin E Oil | [symbol] |
| Vitamin K Oil | [symbol] |
| Peppermint Oil | [symbol] |

### EXAMPLES

The following examples more specifically illustrate formulations for the soothing lip mixture according to various embodiments described above. These examples should in no way be construed as limiting the scope of the present technology.

### Example 1

Table 1, below, shows the composition of an illustrative formulation of the present soothing lip composition. The amounts shown in these tables represent the weight percentage of the particular component based on the total weight of the composition. Table 1 shows the composition of a base formulation used to prepare a number of different examples of the present composition.

TABLE 1
Base Formulation

| | Base |
|---|---|
| Ingredient | Formula(wt. %) |
| Beeswax | [symbol] |
| Shea Butter | [symbol] |
| Mango Butter | [symbol] |
| Sunflower Seed Oil | [symbol] |
| Liquid Coconut Oil | [symbol] |
| Jojoba Oil | [symbol] |
| Arnica Oil | [symbol] |
| Bromelain Extract | [symbol] |

### Example 2

Table 2, below, shows the composition of an illustrative formulation of the present soothing lip composition. The amounts shown in these tables represent the weight percentage of the particular component based on the total weight of the composition. Table 2 shows the composition of a formulation including optional components.

TABLE 2
Formulation with Optional Components

| | Base |
|---|---|
| Ingredient | Formula(wt. %) |
| Beeswax | [symbol] |
| Shea Butter | [symbol] |
| Mango Butter | [symbol] |
| Sunflower Seed Oil | [symbol] |
| Liquid Coconut Oil | [symbol] |
| Jojoba Oil | [symbol] |
| Arnica Oil | [symbol] |
| Bromelain Extract | [symbol] |
| Elderberry Extract | [symbol] |
| Peppermint Oil | [symbol] |
| Aloe Vera Gel | [symbol] |

While certain embodiments have been illustrated and described, it should be understood that changes and modifications can be made therein in accordance with ordinary skill in the art without departing from the technology in its broader aspects.

The embodiments illustratively described herein may suitably be practiced in the absence of any element or elements, limitation or limitations, not specifically disclosed herein. Thus, for example, the terms “comprising,” “including,” “containing,” shall be read expansively and without limitation. Additionally, the terms and expressions employed herein have been used as terms of description and not of limitation, and there is no intention in the use of such terms and expression of excluding any equivalents of the features shown and described or potions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “and” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Additionally, where features or aspects of the disclosure are described in terms of Markush groups, those skilled in the art will realize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof.

What is claimed is:

1. A lip salve composition comprising:
a wax binder component comprising at least two compounds selected from a C₈-C₃₀ fatty acid, an esterified C₈-C₃₀ fatty acid, and a C₈-C₃₀ fatty alcohol;
a complex fat component comprising at least three different compounds selected from C₈-C₃₀ fatty acids;
an organic compound component comprising at least three compounds selected from 2,5-dimethoxy-p-cymene, cumene, thymol methyl ether, 2,6-diisopropylanisole, decanal, α-isocomene, and 1,2,2,3-tetramethylcyclopent-3-enol; and
an enzymatic component comprising cysteine endopeptidase.

2. The lip salve composition of claim 1 comprising:
about 2 to about 40 wt. % of the wax binder component;
about 20 to about 80 wt. % of the complex fat component; and
about 0.1 to about 20 wt. % of the organic compound component
about 0.01 to about 3.0 wt. % of the enzymatic component.

3. The lip salve composition of claim 1 wherein the wax binder is derived from beeswax.

4. The lip salve composition of claim 1 wherein the complex fat component is derived from at least three compounds selected from shea butter, mango butter, sunflower seed oil, liquid coconut oil, and jojoba oil.

5. The lip salve composition of claim 1 wherein the organic compound component comprises arnica oil.

6. The lip salve composition of claim 1 wherein the enzymatic component is derived from bromelain.

7. The lip salve composition of claim 1 further comprising aloe-emodin, aloetic-acid, anthranol, and barbaloin.

8. The lip salve composition of claim 1, comprising:
a) about 2 to about 40 wt. % beeswax;
b) about 10 to about 75 wt. % shea butter;
c) about 5 to about 25 wt. % mango butter;
d) about 1 to about 15 wt. % sunflower seed oil;
e) about 5 to about 35 wt. % coconut oil;
f) about 2 to about 10 wt. % jojoba oil;
g) about 5 to about 30 wt. % arnica oil; and
h) about 0.01 to about 3 wt. % bromelain extract.

9. The lip salve composition of claim 8 further comprising:
a) about 0.5 to 15 wt. % aloe vera gel;
b) about 0.5 to 10 wt. % vitamin E oil
c) about 0.5 to 10 wt. % peppermint oil.

10. The lip salve composition of claim 1, comprising:
a) about 5-10 wt. % beeswax;
b) about 25-40 wt. % shea butter;
c) about 5-15 wt. % mango butter;
d) about 5-15 wt. % sunflower seed oil;
e) about 20-30 wt. % coconut oil;
f) about 1-3 wt. % jojoba oil;
g) about 1-5 wt. % arnica oil;
h) about 1-3 wt. % bromelain extract,
i) about 1-3 wt. % vitamin E oil, and
j) about 1-5 wt. % peppermint oil.

11. A method of making a composition, the method comprising:
combining a wax binder component and a complex fat component to form a base;
heating the base to about a first temperature;
maintaining the base at the first temperature for at least about 20 minutes and then lowering the temperature of the base to a second temperature to form a homogenized base;
adding to the homogenized base an organic compound component and an enzymatic component; and
cooling the homogenized base to a third temperature to form the composition;
wherein the wax binder comprises at least two compounds selected from a C₈-C₃₀ fatty acid, esterified C₈-C₃₀ fatty acid, and C₈-C₃₀ fatty alcohols; the complex fat component comprises at least three different compounds selected from C₈-C₃₀ fatty acids; the organic compound component comprises at least three compounds selected from 2,5-dimethoxy-p-cymene, cumene, thymol methyl ether, 2,6-diisopropylanisole, decanal, α-isocomene, and 1,2,2,3-tetramethylcyclopent-3-enol; and the enzymatic component comprises cysteine endopeptidase.

12. The method of claim 11 wherein the composition comprises:
about 2-40 wt. % of the wax binder component;
about 20-55 wt. % of the complex fat component; and
about 5-25 wt. % of the organic compound component
about 0.01-2.0 wt. % of the enzymatic component.

13. The method of claim 11, wherein the first temperature is about 175° F., the second temperature is about 150° F., and the third temperature is about 50° F.

14. The method of claim 11, wherein the cooling step is performed by exposing the homogenized base to an environment that is about or below 32° F.

15. The method of claim 14, wherein the homogenized base cools from the second temperature to the third temperature in about 30 minutes or less.

16. The method of claim 11, wherein the wax component is derived from beeswax or the complex fat component is derived from at least three compounds selected from shea butter, mango butter, sunflower seed oil, liquid coconut oil, and jojoba oil.

17. The method of claim 11, wherein the organic compound component is arnica oil and the enzymatic component is bromelain.

18. The method of claim 11, further comprising adding aloe-emodin, aloetic-acid, anthranol, barbaloin, vitamin K, olive oil, or cannabidiol, to the homogenized base.

19. A method of making a lip salve composition, the method comprising:
combining beeswax, shea butter, mango butter, sunflower seed oil, liquid coconut oil, and jojoba oil to form a mixture;
heating the base to about 175° F., and then maintaining the temperature of the mixture at about 175° F. for at least about 20 minutes;
reducing the temperature of the mixture to about 150° F.;
adding arnica oil and bromelain extract to the mixture;
and then setting the mixture in a freezing environment to rapidly cool to the mixture to about 50° F.

20. The method of claim 19 further comprising adding a secondary component to the mixture;
wherein the secondary component comprises at least one component selected from the group consisting of aloe vera gel, vitamin E, and peppermint oil.

21. A method of treating pain, the method comprising:
administering to a person in need of treatment a therapeutically effective amount of a composition,
wherein the composition comprises:
a wax binder component comprising at least two compounds selected from a C₈-C₃₀ fatty acid, an esterified C₈-C₃₀ fatty acid, and a C₈-C₃₀ fatty alcohol;
a complex fat component comprising at least three different compounds selected from C₈-C₃₀ fatty acids;
an organic compound component comprising at least three compounds selected from 2,5-dimethoxy-p-cymene, cumene, thymol methyl ether, 2,6-diisopropylanisole, decanal, α-isocomene, and 1,2,2,3-tetramethylcyclopent-3-enol; and
an enzymatic component comprising cysteine endopeptidase.
